(19) United States
(12) Patent Application Publication
(10) Pub. No.: US 2013/0122078 A1
(43) Pub. Date: May 16, 2013

(54) COLLAGEN-CONTAINING CELL CARRIER

(71) Applicant: Eberhard-Karls-Universitaet Tuebingen Universitaetsklinikum, Tuebingen (DE)

(72) Inventors: Lothar JUST, HECHINGEN (DE); Timo SCHMIDT, STUTTGART (DE); Franz MASER, MANNHEIM (DE)

(73) Assignee: Eberhard-Karls-Universitaet Tuebingen Universitaetsklinikum, Tuebingen (DE)

(21) Appl. No.: 13/734,120

(22) Filed: Jan. 4, 2013

Related U.S. Application Data
(62) Division of application No. 12/705,682, filed on Feb. 15, 2010.
Application No. 12/705,682 is a continuation of application No. PCT/EP2008/006660, filed on Aug. 13, 2008.

(30) Foreign Application Priority Data
Aug. 20, 2007 (DE) 10 2007 040 370.6

(51) Int. Cl. A61K 47/42

(52) U.S. Cl. CPC A61K 47/42
USPC 424/443; 424/93.7

(57) ABSTRACT

The present invention relates to a method for the implantation of biological material into an organism.

### CROSS-REFERENCE TO RELATED APPLICATIONS

This application is a divisional of U.S. application Ser. No. 12/705,682, filed Feb. 15, 2010, which is a continuation of International Patent Application PCT/EP2008/006660 filed on Aug. 13, 2008 and designating the United States, which was published in German, and claims priority of German Patent Application DE 10 2007 040 370.6 filed on Aug. 20, 2007. The entire contents of these priority applications are incorporated herein by reference.

### FIELD OF THE INVENTION

The present invention relates to the use of a collagen-containing composition for the cultivation of biological cells, in particular to a method for the cultivation of biological cells, a method for the implantation of biological material into an organism and a method for the improvement of a composition in its suitability for the cultivation of biological cells.

### BACKGROUND OF THE INVENTION

Carriers for the cultivation of biological cells are generally known in the art. Such carriers are often referred to as matrix or scaffold. These carriers provide the breeding ground or, in general, the basis on which the cells grow in cell culture.

Collagen-containing compounds represent the best-known type of cell carriers. Collagen, as an animal protein of the extracellular matrix, belongs to the scleroproteins and is usually water-insoluble and fibrous in structure. It is one of the main components in the structure of connective tissues, e.g. skin, blood vessels, ligaments, tendons and cartilage, and in the structure of bones and teeth. Because of these properties, collagen-based biomaterials from animal sources have already been used in medicine for several years now. Especially in the clinically applicable products for hemostasis, as a replacement for dura or in different areas of plastic surgery, collagens were able to establish themselves as a carrier material. These collagens, of which, to date, 28 different types have been identified and at least 10 additional proteins with collagen-like domains have been registered, show only marginal difference between the individual species. As a distinct identification pattern has not been discovered and as their enzyme-based degradation does not produce any toxic degradation products, collagens are considered biocompatible.

The collagen matrixes so far offered on the market show a very high variance with respect to their properties. Thus, it was found out that, when used in vitro, some of the collagen matrixes used for the cultivation of biological cells cause inflammatory reactions resulting in catabolic metabolism processes. Another disadvantage of the collagen matrixes currently available is that they are very thick—usually exceeding 200 μm—and that they can, thus, not be examined under the microscope. In addition to this, the collagen matrixes offered for the cultivation of biological cells have, so far, been very expensive.

An alternative carrier for the cultivation of cells are the so-called hydrogels. A hydrogel is a water-containing but water-insoluble polymer, whose molecules are chemically (e.g. through covalent or ionic bonds) or physically (e.g. by means interlinking the polymer chains) linked to produce a three-dimensional network. Due to integral hydrophilic polymer components, they expand in water under a considerable intake of volume but without losing their material cohesion. However, the disadvantage of hydrogels is that, given their enormous water-retaining capacity, they are mechanically unstable.

Furthermore, the hydrogels often condense during the colonisation with cells. Therefore, hydrogels are very limited in their use.

The so-called hyaluronic acid represents another matrix suitable for the colonisation with biological cells. This matrix consists of macromolecules of the matrix of cartilage, which is why, in the unmodified form, it shows a high biocompatibility. The molecule chains have to be linked in order to generate a suitable structure with a sufficient mechanical resilience. This is done by means of esterification with alcohol, which can lead to a reduction in biocompatibility.

The so-called alginate represents another scaffold. This concerns a copolymer obtained from brown algae, which consists of L-guluronic acid and D-mannuronic acid. By adding EDTA and/or Na-citrate, the product can be gelatinised or liquefied, which gives the alginate similar properties for the cultivation of cells as those already described for the collagen-based gels, however with improved resuspension possibilities for cellular and molecular biological analyses. Despite the advantages over other carrier matrixes and the good properties of the material in vitro, it was unsuccessful when used in vivo. In vivo, the substance is hard to absorb and causes considerable immune and foreign-body reactions. Therefore, alginate is not yet suitable to be used as a carrier material for human implants.

Another carrier material for biological cells is agarose. It behaves in a similar way to alginate. Agarose consists of two saccharide chains and is obtained from the cell walls of red algae. Like alginate, it causes immune and foreign-body reactions when used in vivo so that agarose cannot yet be used for human implants.

Other scaffolds are based on fibrin. This concerns a globular plasma protein, which, due to its ability of “meshing” polymerisation, amongst other things causes the blood to clot. However, also fibrin has up to now not withstood the test for use in vivo. The use of fibrin as a cell carrier is largely unexplored and must still be extensively evaluated. Furthermore, fibrin is very expensive.

Other matrixes used for the cultivation of biological cells are based on chitin or chitosan. Chitin forms the basic material for the production of chitosan. To this end, the acetyl groups of chitin are chemically or enzymatically split off. Both chitin and chitosan are biopolymers that are not separated by a precisely defined crossover. Usually chitosan is being referred to, when the degree of deacetylation is higher than 40-50% and the compound is soluble in organic acids. However, chitin and chitosan are very limited in their applicability and likewise have not yet proven themselves when used for the cultivation of cells. Chitin is not a material produced inside the body and, therefore, it is constantly a foreign body in the organism. The use of chitin as a cell carrier still has to be fundamentally researched.

At present, there are also numerous synthetic scaffolds being tested. Included in this are the polymers polylactide (PLA) and polyglycolide (PGA) as well as poly-L-lactic acid PPLA (also referred to as “bioglass”). A special characteristic of PLA and PGA is their low solubility in aqueous media that only improves through the degradation of the polymer chain, i.e. hydrolysis, to low-molecular oligomers or monomers, thus leading to the erosion of these materials. However, it has been shown that these polymers are not suitable for the cultivation of biological material. Through spontaneous hydrolysis, absorbable polymers disintegrate and produce organic acids. Osteoblasts differentiate in acid settings so that a higher quantity of these polymers can lead to bone destruction rather than build bones.

### SUMMARY OF THE INVENTION

Against this background, the object underlying the invention is to provide a new composition for the cultivation of biological cells that avoids the disadvantages known for the cell carriers of the art. In particular, a composition should be provided that can be produced economically on a large scale and at a constant high quality.

This object is solved through (1) the provision of a composition suitable for use as a carrier for biological material, comprising the following parameters:

| | |
|---|---|
| Collagen [wt. -%]: | approx. 30 to 80, |
| Amide nitrogen [wt. -%]: | approx. 0.06 to 0.6, |
| Polyol [wt. -%]: | approx. 0 to 50, |
| Fat [wt. -%]: | approx. 0 to 20, |
| Ash [wt. -%]: | approx. 0 to 10, |
| Water [wt. -%]: | approx. 5 to 40, |
| pH value: | approx. 3 to 10, |
| Weight per unit area [g/m²]: | approx. 10 to 100, |
| Tensile strength [N/mm²]: | approx. 0.5 to 100, or 20 to 100, |

(2) contacting said biological material with said composition, and (3) incubating said composition with said biological material under cultivation conditions.

Such a composition has already been made commercially available in form of a film by Naturin GmbH & Co. KG, Badeniastrasse 13, Weinheim. The reference numbers assigned to these films by Naturin are, for example: 400011899, 400023747, 400024203, 400026193, 400019485, 400000084 and 400000109.

This finding was surprising. Until now, this compound was used exclusively in the food industry, for example in the area of ham production as a separating film between net and meat. Above all, it was not expected that such compositions were particularly suitable for the cultivation of biological material.

The composition according to the invention can be reproduced on a large scale and is of a constant high quality. It stands out due to its good biocompatibility, its very thin film thickness, its relatively high transparency and its high mechanical stability, resulting in a wide range of applications.

Furthermore, it is preferred when glycerin or sorbite are used as polyol, which is provided in a concentration of 0 to 50 wt.-% (glycerin), and/or 0 to 40 wt.-% (sorbite).

This measure has the advantage that polyols are used that have particularly proven themselves in the specified concentration as wetting agent or a water-bonding agent to prevent drying-up.

According to a particular configuration the composition comprises the following parameters:

| | |
|---|---|
| Collagen [wt. -%]: | approx. 50 to 70, |
| Amide nitrogen [wt. -%]: | approx. 0.14 to 0.4, |
| Glycerin [wt. -%]: | approx. 12 to 35, |
| Fat [wt. -%]: | approx. 3 to 7, |
| Sorbite [wt. -%]: | approx. 0 to 20, |
| Ash [wt. -%]: | approx. 0.5 to 3, |
| Water [wt. -%]: | approx. 12 to 18, |
| pH value: | approx. 5.5 to 8, |
| Weight per unit area [g/m²]: | approx. 20 to 40, |
| Tensile strength [N/mm²]: | approx. 5 to 25, or 40 to 80. |

The concentrations of the individual ingredients were further optimized with this method so that the composition is further improved in its suitability for the cultivation of biological cells.

To this end, it is preferred that the fat is essentially vegetable oil.

The use of vegetable oil to preserve the composition according to the invention has turned out to be particularly advantageous. Due to the increased elasticity, vegetable oil clearly widens the range of the composition's application. The use of vegetable oils can also prevent rancidity, thus facilitating the manufacture and storage of the composition. It is clear that a minimal amount of residual animal fat does not offset the advantages of the vegetable oil.

According to a particularly preferred configuration, the pH value of the composition according to the invention is approx. 5.0 to 8.0, preferably 6.8 to 8.0, more preferably approx. 7.0 to 8, most preferably approx. 7.2 to 7.5.

As the inventors have found out, the cultivation of biological cells is particularly successful at the specified pH values. Thus, the composition shows a pH value that lies in the physiological area and, therefore, provides a setting that broadly resembles the natural environment of the biological cells. The compositions made commercially available by, for example, Naturin GmbH & Co. KG, usually have a pH value of approx. 4.8. In such acidic environments, for example, the cultivation of biological material sensitive to acids is not possible. The desired pH value can be adjusted through the incubation of the composition in, for example, calcium or magnesium-containing phosphate buffers, whereby other traditional buffers well-known persons skilled in the art are equally suitable.

Against this background, another object of the present invention is a method to improve a composition with following parameters:

| | |
|---|---|
| Collagen [wt. -%]: | approx. 30 to 80, |
| Amide nitrogen [wt. -%]: | approx. 0.06 to 0.6, |
| Polyol [wt. -%]: | approx. 0 to 50, |
| Fat [wt. -%]: | approx. 0 to 20, |
| Ash [wt. -%]: | approx. 0 to 10, |
| Water [wt. -%]: | approx. 5 to 40, |
| pH Value: | approx. 3 to 10, |
| Weight per unit area [g/m²]: | approx. 10 to 100, |
| Tensile strength [N/mm²]: | approx. 0.5 to 100, or 20 to 100, |

in its suitability for the cultivation of biological cells, which includes the following steps:
(1) Provision of the composition, and
(2) Adjusting the pH value to approx. 5.0 or 8.0, preferably to approx. 6.8 to 8.0, more preferably to approx. 7.0 to 7.8, most preferably at approx. 7.2 to 7.5.

Thanks to this “optimisation method”, commercially available films like, for example, those offered by Naturin GmbH & Co. KG, show a clearly improved suitability for use as cell culture carriers. Step (2) is preferably carried out as an incubation of the composition in a buffer solution with a pH value of approx. 7.2 to 7.5.

The optimisation method preferably comprises an additional step (3), during which the composition is incubated with highly fat-soluble substances.

This measure has the advantage that, for example, fat-soluble substances are extracted by using 100-% acetone. The quality of the composition for the cultivation of biological materials can thus be further improved.

In addition, during the optimisation method according to the invention, it is preferred that step (3) includes a step (3.1), during which the composition is washed in the buffer solution.

During this step (3.1), the remaining highly fat-soluble substances and, if necessary, other contaminating residuals are removed so that the membrane is then ready for use or can be further processed or treated.

Preferably, the optimisation method according to the invention also comprises an additional step (4), during which the drying of the composition takes place.

This measure has the advantage that it serves to obtain a product that is simple to handle and that can be stored almost indefinitely.

Following a preferred configuration, the composition according to the invention is configured as a flat film.

Thanks to this measure, the composition is provided in a form that is particularly suitable for both cell cultivation applications and the implantation of biological material in an organism. The composition according to the invention configured as a film can be easily cut or pressed to any shape or size.

Against this background, the composition is preferably configured as a carrier or matrix or “scaffold”, respectively, for cell cultivation applications or as a carrier for the implantation of biological material into an organism, preferably of stem and precursor cells or specific tissue cells. Due to its biocompatibility and its adherence-supporting properties, it can be used for immobilizing cells. This can be of great relevance in the field of regenerative medicine, for example, in the development of cell cultures for ligaments, tendons, bones and cartilage. Furthermore, however, it can also be used for other tissues. Therefore, it is also suitable for use as a wound dressing. Additionally, the composition can be used for example in the field of cosmetics as skin dressing. Furthermore, due to the chemical compositions of the collagens, it is especially suitable for interlinking cell-affecting components, like for example, growth factors.

A particular advantage is the fact that the flat film according to the invention adheres onto flat synthetic surfaces after drying without any extra help. As a result, the film, for example with the cell culture's plastic surface, produces a bubble-free unit, which remains intact even during low-strain cell cultivation. Thus, cell-affecting gluing and affixing aids in the cell culture can be eliminated. For specific applications, however, this bonding can be dissolved using mechanical tools, e.g. tweezers, without causing damage and the film can be removed from cell culture dish together with the cells growing on it.

As an alternative, the composition according to the invention is configured as a tubular casing.

This configuration is particularly suitable for use as a cell and substance reservoir for implantation tests.

In dry conditions, the flat film or tubular casing according to the invention comprises a thickness of approx. 5 to 200 μm, preferably 10 to 100 μm, more preferably 15 to 30 μm, more preferably approx. 20 μm, and most preferably approx. 15 μm.

This measure has the advantage that a particularly thin film or casing is provided that is transparent and can also be examined under the microscope. This is not the case as regards the collagen-based cell carriers known in current state-of-the-art technology. According to the invention, the thickness is determined in a dried condition. The term “dried” here is equivalent to air-dried so that an absolute residual humidity remains, amounting to approx. 10% to 15%.

According to a preferred further development, the composition is radiation-sterilized, which is preferably carried out by means of ionising radiation or, more preferably, by means of beta and/or gamma-radiation.

This measure has the advantage that contaminating organisms are killed and contaminations of the biological material to be cultivated are broadly avoided. The radiation-sterilization method offers the advantage that the heat-sensitive collagen remains undamaged.

Against this background the optimisation method according to the invention comprises the additional step (5) during which the radiation-sterilization of the compound takes place.

Furthermore, it is preferred that the composition according to the invention is also furnished with a dye, preferably a fluorescence-absorbing dye.

For the purpose of in-vitro diagnostics, the composition can be differently coloured. In doing process, fluorescence-coloured cells that have penetrated through the film can be established during so-called penetration tests for pharmacological, physiological or cell-biological tests because a, for example, fluorescence-absorbing colour of the composition covers the fluorescent cells that have not been penetrated. Then, only the penetrated cells glow during the fluorescence microscopic test.

Another object of the present invention is a method for the implantation of biological material into an organism, comprising the following steps:
(1) Providing a composition suitable for use as a carrier for biological material,
(2) Contacting the biological material with the composition, and
(3) Introducing the biological material in contact with the composition into an organism,

whereby the composition above-mentioned in connection with the application according to the invention described is used as the composition.

The composition according to the invention can also be used for the determination of the invasion and metastasising potential of tumor cells.

Common in-vitro test methods for the invasion and metastasising potential of tumor cells are based on the following principle: the penetration capacity of tumor cells is measured through a perforated and non-degradable synthetic film. In in vitro tests, this system, for example, serves to measure the effect of anti-carcinogenic pharmaceuticals on the penetration capacity of the cells. The invasiveness of a tumor cell, however, does not only depend on the migration capacity through gaps (pores) in the connective tissue but also on the cells' ability to enzymatically degrade or rebuild the connective tissue's components that mainly consist of the collagens. Due to its low thickness, homogeneity and standardised production, the biological collagen film could serve to develop a new pharma-test system for the determination of the proteolytic capacity of cultivated cells. For this system, the cells are cultivated in a two-chamber system. The two chambers are separated using the collagen film. The cells to be examined are cultivated in the upper chamber on the film. Following a corresponding cultivation period, the number of cells migrated through the collagen membrane onto the bottom side of the membrane is quantified. Therefore, the use of the composition according to the invention opens up a new area in in-vitro diagnostics.

The composition according to the invention can also be used to determine the proteolytic activity of non-tumor cells and their invasion potential, e.g. as a vitality test for stem cells.

It is clear that the characteristics both mentioned above and explained in the following are not only applicable in the respective given combination but also in other combinations or in an isolated approach, without departing the scope of the present invention.

The invention is now explained in more detail on the basis of embodiments from which further criteria and advantages as well as characteristics of the invention arise. Reference is made to the figures attached.

### BRIEF DESCRIPTION OF THE DRAWINGS

FIG. 1 shows a collagen inlay with a thickness of 20 μm for a cell culture panel with 6 cavities (A), a tubular casing according to the invention as a cell reservoir for implantation tests (B, B′).

FIG. 2 shows the result of a BrdU proliferation assay on human mesenchymal stem cells (hMSC). Immune-cytochemical analysis of the BrdU-positive cells. The cells were cultivated on a conventional synthetic culture surface (A, A′) and on the collagen matrix according to the invention and incubated with BrdU for one hour.

FIG. 3 shows the result of the BrdU proliferation assay (A) and the MTT test (B) on human mesenchymal stem cells (hMSC) that were cultivated on a collagen matrix and a conventional synthetic culture surface. The average values are presented with the respective standard deviations.

FIG. 4 shows a top view of a mineralised collagen matrix that was cultivated together with hMSCs under osteogenic differentiation conditions (A, A′); alkaline phosphate activity from embryonal (E18) murine osteoblasts from the cranial calotte after a two-week cultivation phase on the collagen matrix (B, B′).

FIG. 5 shows a paraffin cross section through the collagen matrix that was cultivated with embryonal (E18) murine osteogenic progenitors from the cranial calotte under proliferation conditions (A, A′) and osteogenic differentiation conditions (B, B′). Figures B and B′ clarify the continuous mineralization of the 3-dimensional matrix. The integration and penetration capacities depend on the cell type.

FIG. 6 shows the implantation of a cell-free tubular casing according to the invention in a C57/BL6-murine (A) and the implanted matrix after 6 weeks in the nude murine (B).

FIG. 7 shows the tubular film according to the invention directly prior to the implantation that colonised for one day with hMSCs (A) as well as the explanted implant grown into the connective tissue after 6 weeks (B).

FIG. 8 shows the tubular film according to the invention following the explantation after 6 weeks in a C57/BL6-murine in an enlarged presentation. The blood vessels that pervade the collagen film are clearly recognisable.

FIG. 9 shows the He-colouring on a paraffin cut that was made from an explanted implant.

### DESCRIPTION OF PREFERRED EMBODIMENTS

1. Collagen-Containing Composition

The inventors used seven films made commercially available by Naturin GmbH & Co. KG and found out that they were suitable for the application according to the invention. The parameters of the commercially available films tested are listed in the following Table 1:

TABLE 1
parameters of the collagen-based films made available by Naturin

| Composition # | 1 | 2 | 3 | 4 | 5 | 6 | 7 |
|---|---|---|---|---|---|---|---|
| Reference no. | 400011899 | 400023747 | 400024203 | 400026193 | 400019485 | 400000084 | 400000109 |
| Configuration of the | flat film | tubular | tubular | tubular | tubular | tubular | tubular |
| composition | | casing | casing | casing | casing | casing | casing |
| | | Ø 110 mm | Ø 140 mm | Ø 60 mm | Ø 40 mm | Ø 65 mm | Ø 115 mm |
| Collagen [wt.-%] | 65 | 76 | 79 | 76 | 79 | 77 | 77 |
| Water | 15 | 12 | 12 | 12 | 12 | 12 | 12 |
| Glycerine [wt.-%] | 15 | 10 | 7 | 10 | 7 | 4 | 4 |
| Fat | | | | | | | |
| acetoglyceride [wt.-%] | 4 | — | — | — | — | — | — |
| vegetable oil [wt.-%] | — | 1 | — | 1 | 1 | 1 | 1 |
| Ash [wt.-%] | 1 | 1 | — | 1 | 1 | 1 | 1 |
| pH value | 5.1 | 3.4 | 3.4 | 3.4 | 3.4 | 4.8 | 4.8 |
| Thickness [μm] | 20 | 110 | 25 | 82 | 67 | 100 | 115 |
| Examination under the | good | possible | not | fluorescence | fluorescence | not | fluorescence |
| Microscope (transmitted | | | possible | | | possible | |
| light/fluorescence) | | | | | | | |

1.1 Production of Compositions in Film form According to the Invention

Bovine hide splits serve as the starting material for the production of the composition according to the invention in film form, which, with regard to their traceability and the hygiene standards, meet the requirements specified in Regulation (EC) No. 853/2004.

These bovine hide splits are roughly mechanically pre-cut and in several method steps at first washed with water and subsequently decomposed using alkaline. The level of decomposition can be varied and depends on factors such as the duration of the treatment, the concentration of the alkaline medium (pH value) and the temperature. Lime water, sodium hydroxide solution or a mixture of these two components are normally used to set the alkaline medium. However, other alkaline combinations are equally suitable. The alkaline treatment is carried out at a pH value of, for example, 12.5 and can range, for example, from 15 hours to over 150 hours, depending on the intended intensity of the hide decomposition. Amide nitrogen proved as a possible parameter for the analytical tracing of the level of decomposition of the collagen tissue: the more intensive the decomposition, the lower the amide nitrogen.

After reaching the desired level of decomposition, acid is added and, subsequently, water is repeatedly used for rinsing. The acidification is usually done using hydrochloric acid over a period of 6 to 10 hours, reaching a pH value of <2, preferably <1. The use of other acids is also possible. The pH value is subsequently increased from 2.6 to 3.3 by means of numerous downstream rinsing procedures using water.

The resulting “collagen callosities” are then mechanically processed by means of mincing and pressing the minced material through perforated discs with gradually smaller aperture sizes into a gel-like, viscoelastic matter.

1.1.1 Development as a Flat Film (Composition No. 1)

This “concentrated” collagen mass is transferred into an agitator into which the glycerine, water and acid are added. At the same time, the pH value is adjusted to preferably 2.6-3.2 and the percentage of dry collagen is adjusted between 1.6 wt.-% and 2.5 wt.-%. The mixture subsequently passes through a homogeniser, is aerated and subsequently poured through a slit nozzle onto a conveyor belt, on which the resulting gel film passes through a tunnel drier. Before entering the drier, it is fumigated preferably using ammonia gas, thus raising the pH value of the gel. At the end of the drier, the dried film passes through a re-hydration zone before it is wrapped up.

1.2 Development as a Tubular Casing (Compositions No. 2 to 7)

The viscoelastic collagen mass from 1.1 is transferred into a moulding mixer, into which glycerine is added depending on the formula. The pH value and the percentage of dry matter are adjusted at the same time as the water and acid are added.

The homogenous mass is subsequently extruded through a ring slotted nozzle, thereby producing an endless tubular casing. A simultaneous injection of supporting air protects the tubular casing against collapsing.

The transport of the blown tubular casing through the extrusion line proceeds differently in detail depending on the type of intestine to be produced. In principle, there is the possibility to pass through chemical-containing showers and drying segments in a variable sequence. At the end of the extrusion line the dried tubular casing is laid flat between squeegees and wound up on spools in this condition.

The tubular films obtained then undergo a thermal treatment, whereby they acquire the required mechanical stability for their later use. Compositions according to the invention in film or tubular form can also be made on the basis of other collagen sources, whereby the processing of the collagen gel may differ in its detail from the preceding descriptions. Based on pig hide collagen, for example, a suitable way has to be found to reduce the fat content, which, for example, is described in DE 100 60 643 and EP 1 423 016. The use of natural intestines to produce a collagen matter is, for example, described in ES 2 017 564. These documents are incorporated in the disclosure of the current application by reference.

1.2 Adjusting the pH Value

The pH value is adjusted through the use of a calcium and magnesium containing phosphate buffer [phosphate buffered saline (PBS) with Ca⁺⁺ and Mg⁺⁺ (PAA H15-001)] that adjusts the pH value of the collagen-based film in the physiological area of pH 7.2 to pH 7.5. To this end, the collagen film is washed with the buffer system by means of agitation for 5 days. The buffer is exchanged twice a day.

Alternatively, the collagen membrane can also be immersed for an hour in a phosphate buffer containing glycerine with a pH value of 7.3 (phosphate buffer: 15.6 g of KH₂PO₄, 71.3 g of Na₂HPO₄×2H₂O and 492.9 g of glycerine are dissolved into 7722 g of distilled water). Afterwards, the processed film is left to drain and placed into a tenter frame, where it dries overnight at room temperature.

1.3 Further Optional Processing

After a short equilibration in distilled water, the collagen membrane is processed with 100% acetone to extract the fat-soluble substances and break down the water-soluble proteins. After the removal of the acetone, the dried membrane is washed at negative pressure 3 times for one hour each using the calcium and magnesium-containing phosphate buffer (in g/l: KCl 0.2; KH₂PO₄ 0.2; NaCl 8.0; Na₂HPO₄ anhydrous 1.15; CaCl₂-2H₂O in H15-001 0.132; MgCl₂-2H₂O in H15-001 0.1). To eliminate the buffer salt, the washing procedure is repeated 3 times for one hour each in distilled water.

1.4 Drying

The available membrane or film is dried. This can be done in a drying cabinet at 60° C., whereby a humidity value of <5%, for example 3%, can be reached. The membrane can also be dried at room temperature simply by leaving it to dry in the air so that it will finally adjust itself to the relative air humidity depending on the balancing humidity of the membrane or film that usually amounts to between approx. 8 wt.-% and approx. 13 wt.-%.

1.5 Shaping and Radiation Sterilisation

The dried collagen membranes or films obtained can be cut in any way or punched, e.g. in DIN A5 sheets. These sheets are then sterilised by means of beta or gamma irradiation at 25 kGy or 50 kGy.

The collagen film can, for example, be finished as an insert for synthetic deepening cups of any construction type, for example microtitre plates, or produced as preferably seamless tubular casings with a diameter of <2 mm, approx. 12 mm up to several centimetres. Thermal welding or gluing the film is also possible.

1.6 Parameters of the Produced Composition According to the Invention in Film Form

The parameters of different flat collagen films are presented in the following table 2, which were reached in accordance with the procedures described in 1.1.1, whereby the steps described in accordance with section 1.3 were not carried out.

TABLE 2
parameters of the produced films based on collagen

| Sample | A | B | C | D | E | F | G |
|---|---|---|---|---|---|---|---|
| Collagen [wt.-%] | 58 | 61 | 61 | 61 | 55 | 55 | 55 |
| Amide nitrogen | 28 | 37 | 37 | 37 | 31 | 31 | 31 |
| [mmol/100 g | | | | | | | |
| dry collagen] | | | | | | | |
| Glycerine [wt.-%] | 16 | 25 | 25 | 25 | 30 | 30 | 30 |
| vegetable oil [wt.-%] | 5 | 0 | 0 | 0 | 0 | 0 | 0 |
| Sorbite [wt.-%] | 3 | 0 | 0 | 0 | 0 | 0 | 0 |
| Ash(600° C.); [wt.-%] | 2 | 1 | 1 | 1 | 1 | 1 | 1 |
| Water content | 16 | 13 | 13 | 13 | 14 | 14 | 14 |
| pH value | 5.2 | 7.0 | 7.0 | 7.0 | 7.1 | 7.1 | 7.1 |
| Weight per unit | 32 | 38 | 29 | 23 | 30 | 27.5 | 25 |
| area [g/m²]: | | | | | | | |
| Tensile strength, | 60 | 67 | 61 | 44 | 59 | 54 | 48 |
| length [N/mm²] | | | | | | | |
| Tensile strength, | 52 | 54 | 48 | 38 | 52 | 47 | 43 |
| cross [N/mm²] | | | | | | | |
| Type of | none | (*) | (*) | (*) | (*) | (*) | (*) |
| sterilisation and | | | | | | | |
| dose | | | | | | | |

(*) For every sample from 1 to 6, there were 5 sub-tests: without sterilisation (a), beta-radiation 25 kGy (b), beta-radiation 50 kGy (c), gamma-radiation 25 kGy (d) and gamma-radiation 50 kGy (e)

The following methods of analysis were applied:

Collagen over hydroxiproline regulation/amide nitrogen analogue EP1676595 (Geistlich Söhne AG)/Glycerine over HPLC/vegetable oil through Soxhlet extraction/Sorbite over HPLC/Gravimetric ash after incineration in a muffle furnace for 5 hours at 600° C.)/Gravimetric water content after drying in the drying cabinet at 150° C./pH value by snipping the film into small pieces, inserting the snippets in a 5-% NaCl solution and measuring using a glass electrode after 10 minutes/mass per unit area by weighing a 10 cm×10 cm piece of film with balancing humidity/tensile strength lengthways and across by means of a UTS universal testing machine (model 3/205, UTS Testsysteme GmbH) after air-conditioning at 21° C./60% relative humidity of the punched sample body and a traverse speed of 100 mm/min.

2. Cultivation of Biological Material

2.1 Configuration of the Composition

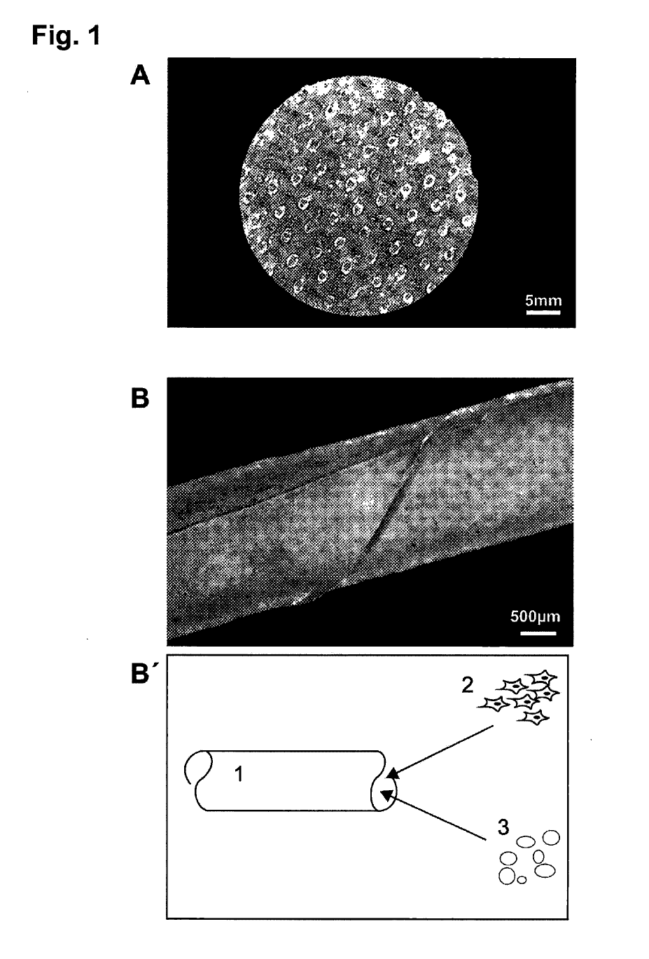

FIG. 1 shows a collagen film according to the invention with a thickness of 20 μm, configured as an insertion for a cavity of cell culture panel (A). A tubular casing is shown in the part illustration (B), which is schematically presented in part illustration (B′). The tubular casing is shown at reference number 1. The cells are shown at reference number 2, which can be placed in the interior of the casing. An active substance or growth factors are shown at reference number 3, which also can be placed in the casing in order to influence the biological cells.

2.2 Proliferation Behaviour of Human Cells

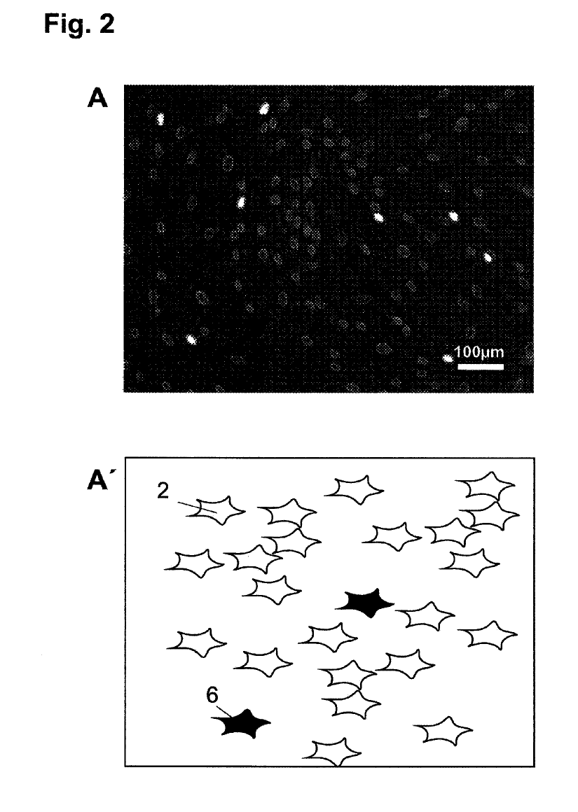

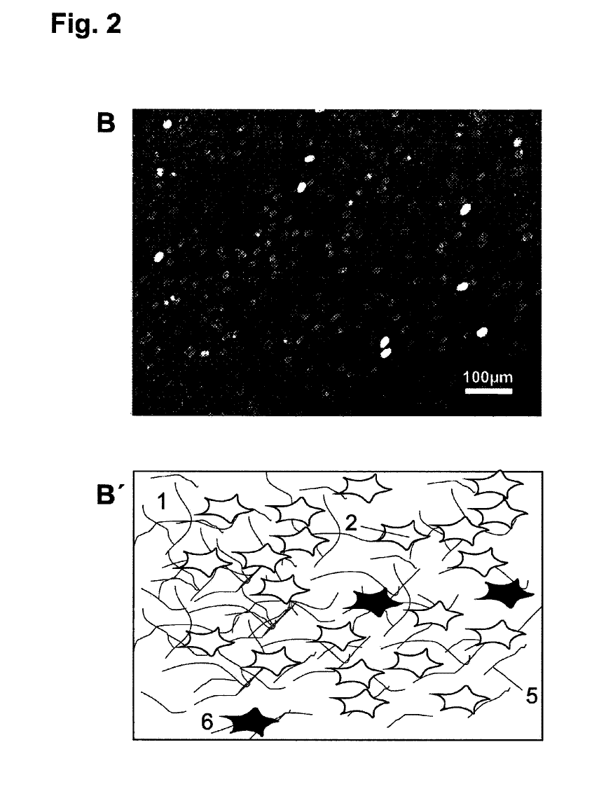

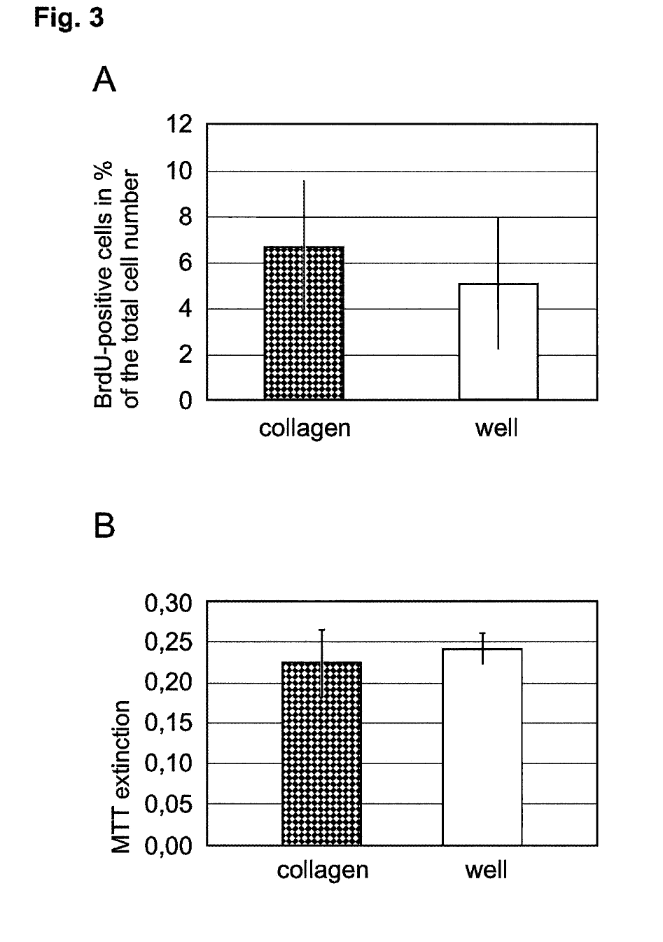

The proliferation behaviour of human cells, mesenchymal stem cells MSC and the human cell line SaOS2 on the collagen film according to the invention did not show any difference in comparison with the conventional cultivation procedures in the plastic culture basin; FIG. 2. The cells were cultivated on a conventional plastic culture surface (A) and on the collagen film according to the invention (B) and incubated with BrdU for one hour. The schematic illustration (B′) shows the collagen film at 1, the cells at 2, the collagen fibres at 5 and the BrdU-positive cells at 6. The statistical evaluation of the BrdU proliferation assay (A) and the MTT vitality test (B) are presented in FIG. 3.

Afterwards, no significant differences are shown between the collagen film according to the invention and the conventional plastic basins.

2.3 Biocompatibility

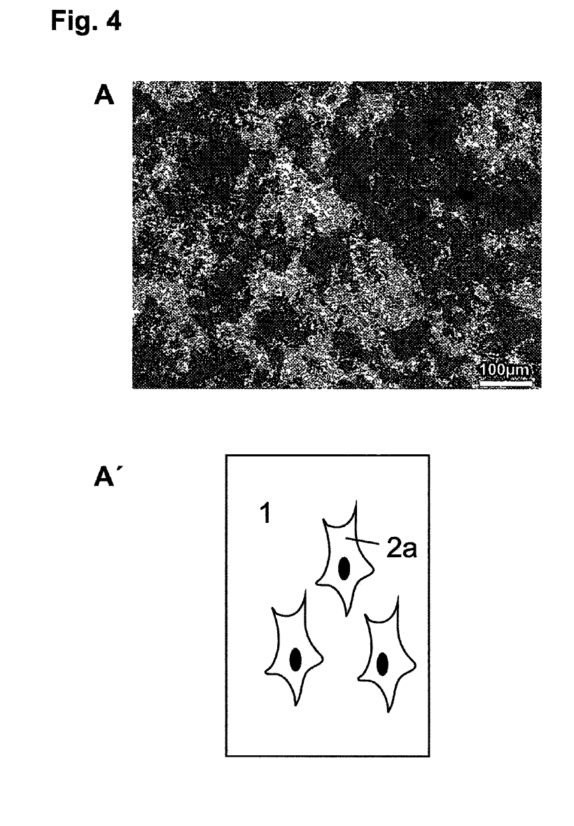

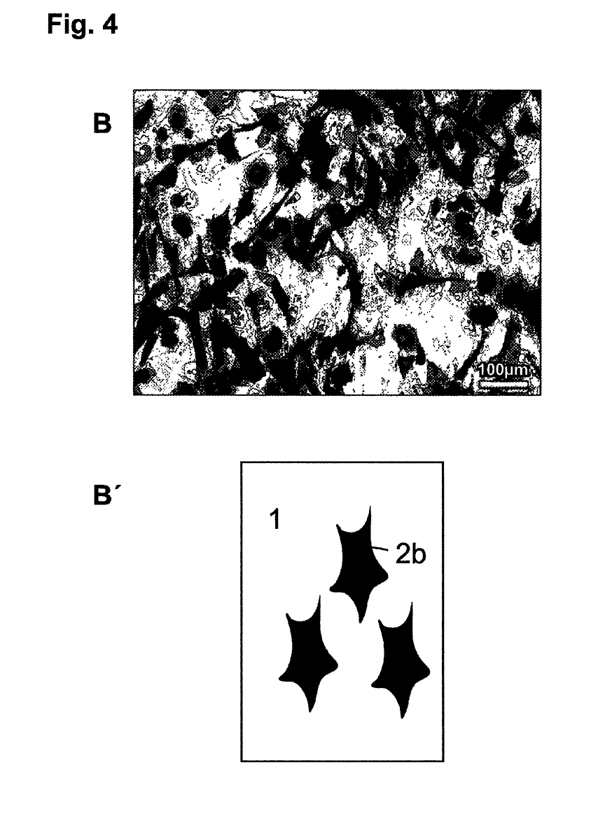

Both embryonal murine progenitors from the cranial calotte and hMSCs were cultivated on this matrix under osteogenic differentiation conditions for the evaluation of the biocompatibility of the collagen film according to the invention. The result is presented in FIG. 4.

Part illustration (A) shows an overview of a mineralised collagen matrix according to the invention, which was cultivated together with hMSCs under osteogenic differentiation conditions. Part illustration (B) shows the alkaline phosphate activity of embryonal murine osteoblasts from the cranial calotte after a 2-week cultivation period on the collagen foil according to the invention. The schematic part illustrations (A) and (B′) show the collagen membrane at 1, the cells at 2a and the cells after detection of the cellular alkaline phosphate activity at 2b.

The detection of the alkaline phosphate activity and the cell-induced mineralization clarifies the differentiation potential of the cultivated cells and, thus, the biocompatibility of the matrix according to the invention.

2.4 Cultivation of Three-Dimensional Tissue Structures

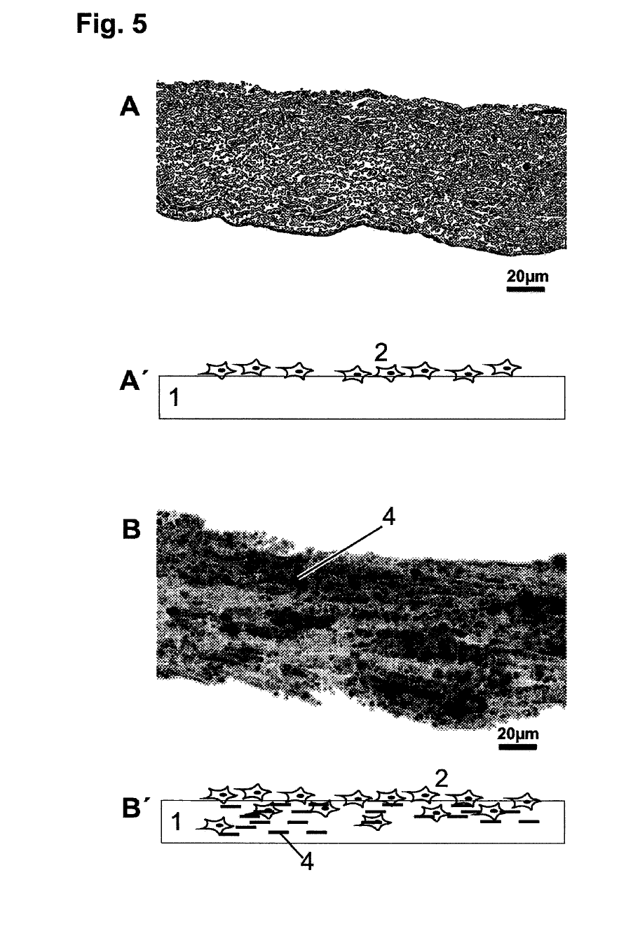

Paraffin cross-sections are made from colonised collagen films. These were histochemically analysed with regard to the mineralization. The result is presented in FIG. 5. Part illustration (A, A′) shows the paraffin cross-section under proliferation conditions, part illustration (B, B′) under osteogenic differentiation conditions. 1 refers to the collagen film, 2 to the cells and 4 to the silver nitrate deposits.

On the one hand, this experiment shows the high mineralization potential and, on the other hand, the integration ability of the cells within the three-dimensional film/matrix. With the help of this matrix according to the invention, the cultivation of three-dimensional tissue structures is conceivable.

2.5 Implantations

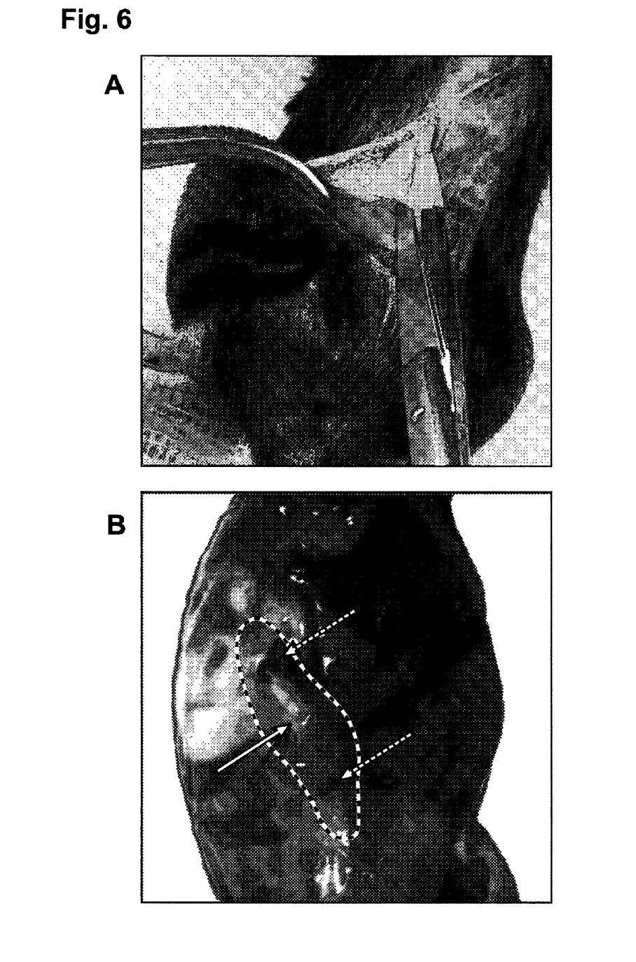

Implantation experiments were carried out on nude and C57/BL6 mice. To this end, cell-loaded tubular casings according to the invention were implanted in the area between the subcutis and the peritoneum. The result of this experiment is shown in FIG. 6. Part illustration (A) shows the implantation and part illustration (B) shows the implanted matrix according to the invention after 6 weeks in the nude mouse. The drawn-through arrow points to the cell-loaded tubular casing according to the invention. With the help of the fixing points, the tubular casing with the inserted non-biodegradable filaments can also be easily located in part illustration (B); dotted arrow. In part illustration (B), the preparation clearly shows the still existing tubular casing according to the invention.

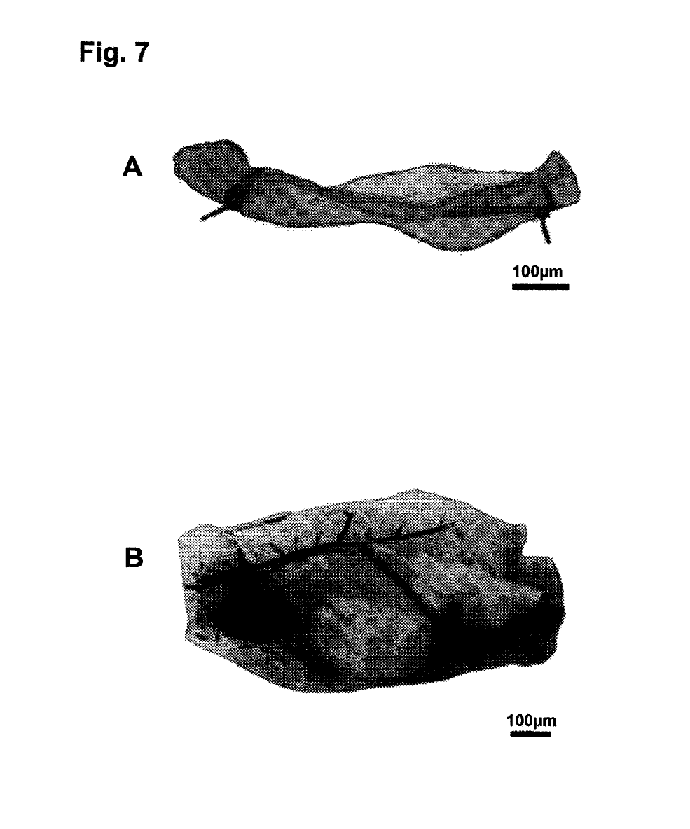

FIG. 7, part illustration (A) shows the tubular casing according to the invention directly prior to the implantation, which was colonised for one day with hMSCs. Part illustration (B) shows the explanted implant grown in the connective tissue after 6 weeks. Thereby, it becomes apparent that even 6 weeks after the implantation the integrity of the tubular casing remains intact despite an incipient absorption process.

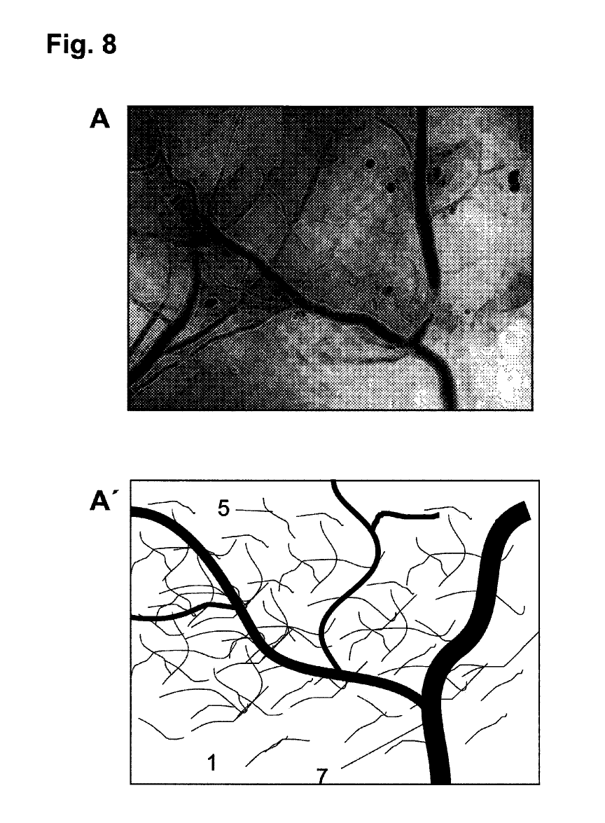

The implant has clearly grown in the connective tissue of the animal and was crossed by blood vessels; see also FIG. 8 (A, A′). The schematic illustration (A′) marks the collagen membranes (1), the cells (2), the collagen fibres (5) and the blood vessels (7).

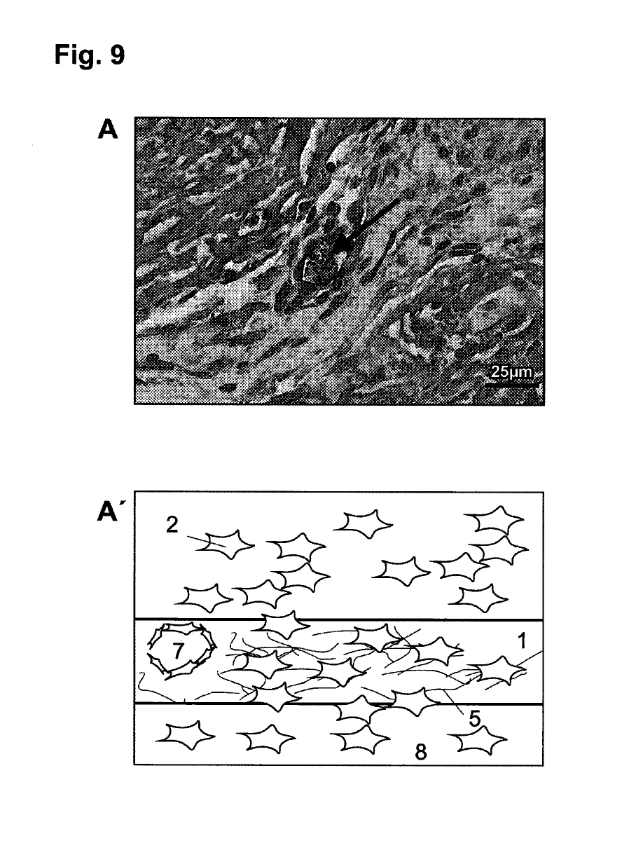

Immune-histological analyses of HE-coloured paraffin cuts show cells that have migrated into the foil according to the invention; see also FIG. 9. A blood vessel can be clearly established in the area of the implants (A, arrow). In the schematic illustration (A) 1 refers to the tubular casing, 2 to the cells, 5 to the collagen fibres, 7 to a blood vessel and 8 to the connective tissue.

3. Conclusion

The inventors could supply a collagen-containing composition, for example in film or casing form, which is reproducible in large-scale manufacturing and which is especially well-suited for the cultivation and generation of biological materials.

1. A method for the implantation of biological material into an organism, comprising the following steps:
(1) Providing a composition suitable for use as a carrier for biological material,
(2) Contacting said biological material with said composition, and
(3) Introducing said biological material in contact with said composition into an organism,
wherein said composition comprises the following parameters:

| | |
|---|---|
| Collagen [wt. -%]: | approx. 30 to 80, |
| Amide nitrogen [wt. -%]: | approx. 0.06 to 0.6, |
| Polyol [wt. -%]: | approx. 0 to 50, |
| Fat [wt. -%]: | approx. 0 to 20, |
| Ash [wt. -%]: | approx. 0 to 10, |
| Water [wt. -%]: | approx. 5 to 40, |
| pH Value: | approx. 3 to 10, |
| Weight per unit area [g/m²]: | approx. 10 to 100, |
| Tensile strength [N/mm²]: | approx. 0.5 to 100. |

2. The method of claim 1, wherein the polyol is selected from:

| | |
|---|---|
| Glycerin [wt. -%]: | approx. 0 to 50, and/or |
| Sorbite [wt. -%]: | approx. 5 to 40. |

3. The method of claim 1, wherein the composition comprises the following parameters:

| | |
|---|---|
| Collagen [wt. -%]: | approx. 50 to 70, |
| Amide nitrogen [wt. -%]: | approx. 0.14 to 0.4, |
| Glycerin [wt. -%]: | approx. 12 to 35, |
| Fat [wt. -%]: | approx. 3 to 7, |
| Sorbite [wt. -%]: | approx. 0 to 20, |
| Ash [wt. -%]: | approx. 0.5 to 3, |
| Water [wt. -%]: | approx. 12 to 18, |
| pH Value: | approx. 5.5 to 8, |
| Weight per unit area [g/m²]: | approx. 20 to 40, |
| Tensile strength [N/mm²]: | approx. 5 to 25. |

4. The method of claim 1, wherein the fat is essentially vegetable oil.

5. The method of claim 1, wherein the pH value is at approx. 5.0 to 8.0.

6. The method of claim 1, wherein the pH value is at approx. 7.2 to 7.5.

7. The method of claim 1, wherein said composition is configured as a flat film.

8. The method of claim 7, wherein said flat film in a dry condition comprises a thickness of approx. 5 to 200 μm.

9. The method of claim 8, wherein said flat film in a dry condition comprises a thickness of approx. 15 μm.

10. The method of claim 1, wherein said composition is configured as a tubular casing.

11. The method of claim 1, wherein said composition is sterilised through radiation before contacting said biological material.

12. The method of claim 11, wherein the radiation sterilisation is carried out using ionising radiation.

13. The method of claim 12, wherein the ionising radiation is beta-radiation and/or gamma-radiation.

14. The method of claim 1, wherein the composition comprises a fluorescence-absorbing dye.

15. The method of claim 1, wherein said biological material comprises stem precursor cells.
